(19) United States
(12) Patent Application Publication
(10) Pub. No.: US 2026/0131482 A1
(43) Pub. Date: May 14, 2026

(54) ADVANCED 3D GRASPER DESIGN

(71) Applicant: CILAG GMBH INTERNATIONAL, Zug (CH)

(72) Inventors: Konstantin G. ZABOTKIN, Mason, OH (US); Austin WISE, Cincinnati, OH (US); Andrew Ross CONWAY, Cincinnati, OH (US)

(73) Assignee: CILAG GMBH INTERNATIONAL, Zug (CH)

(21) Appl. No.: 19/379,408

(22) Filed: Nov. 4, 2025

Related U.S. Application Data
(60) Provisional application No. 63/718,040, filed on Nov. 8, 2024.

(51) Int. Cl. B25J 15/00; A61B 34/30; B25J 15/02

(52) U.S. Cl. CPC B25J 15/0033; A61B 34/30; B25J 15/0233

(57) ABSTRACT

An end effector of a surgical tool is disclosed. The end effector includes a first jaw and a second jaw. The first jaw includes a first jaw body and first grips extending from the first jaw body. The second jaw includes a second jaw body and second grips extending from the second jaw body. The first and second jaws are movable between open and closed configurations. The first and second grips mesh upon transitioning the first and second jaws to the closed configuration to thereby define a non-linear longitudinal interface along the length of the first and second jaws and a non-linear lateral interface along the width of the first and second jaws.

### CROSS-REFERENCE TO RELATED APPLICATIONS

The present application claims priority under 35 U.S.C. §119(e) to U.S. Provisional Application No. 63/718,040, titled “ADVANCED 3D GRASPER DESIGN”, filed November 8, 2024, the disclosure of which is hereby incorporated by reference in its entirety.

### BACKGROUND

The present disclosure is related to surgical tools and, more specifically, to surgical tools with end effectors for grasping and manipulating tissue during a surgical procedure.

During a surgical procedure, robotic surgical tool end effectors, such as tissue graspers, are commonly used to grasp and manipulate the patient tissue. While grasping and manipulating the tissue, there is a chance that the tissue may slip relative to the end effector. One possible solution may be to increase the grip force applied to the tissue by the end effector. However, the amount of grip force that an end effector of a robotic surgical tool can apply to tissue is significantly less compared to the amount of grip force that an end effector of a handheld surgical tool can apply to tissue. Furthermore, increasing the grip force may shorten the life of the surgical instrument.

Accordingly, an improved end effector design is desired to improve tissue grip without increasing grip strength.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 shows a diagram of an example surgical tool that may incorporate certain principles of the present disclosure.

FIG. 2 shows a diagram illustrating the degrees of freedom through which a wrist of a surgical tool may articulate.

FIG. 3 shows an enlarged, isometric view of the distal end of the surgical tool of FIG. 1.

FIG. 4A is an enlarged side view of the jaws of an end effector in a closed configuration and for use with the surgical tool of FIG. 1, in accordance with at least one aspect of the present disclosure.

FIG. 4B is a front view of the end effector of FIG. 4A, in accordance with at least one aspect of the present disclosure.

FIG. 4C is a front view of the end effector of FIG. 4A in an open configuration, in accordance with at least one aspect of the present disclosure.

FIG. 4D is a detailed view of a portion of the end effector of FIG. 4A, in accordance with at least one aspect of the present disclosure.

FIG. 4E is a detailed view of a portion of the end effector of FIG. 4C, in accordance with at least one aspect of the present disclosure.

FIG. 5A is an enlarged side view of the jaws of another end effector in a closed configuration and for use with the surgical tool of FIG. 1, in accordance with at least one aspect of the present disclosure.

FIG. 5B is a front view of the end effector of FIG. 5A in an open configuration, in accordance with at least one aspect of the present disclosure.

### DETAILED DESCRIPTION

The present disclosure is related to surgical tools and, more specifically, to surgical tools with end effectors for grasping and manipulating tissue during a surgical procedure.

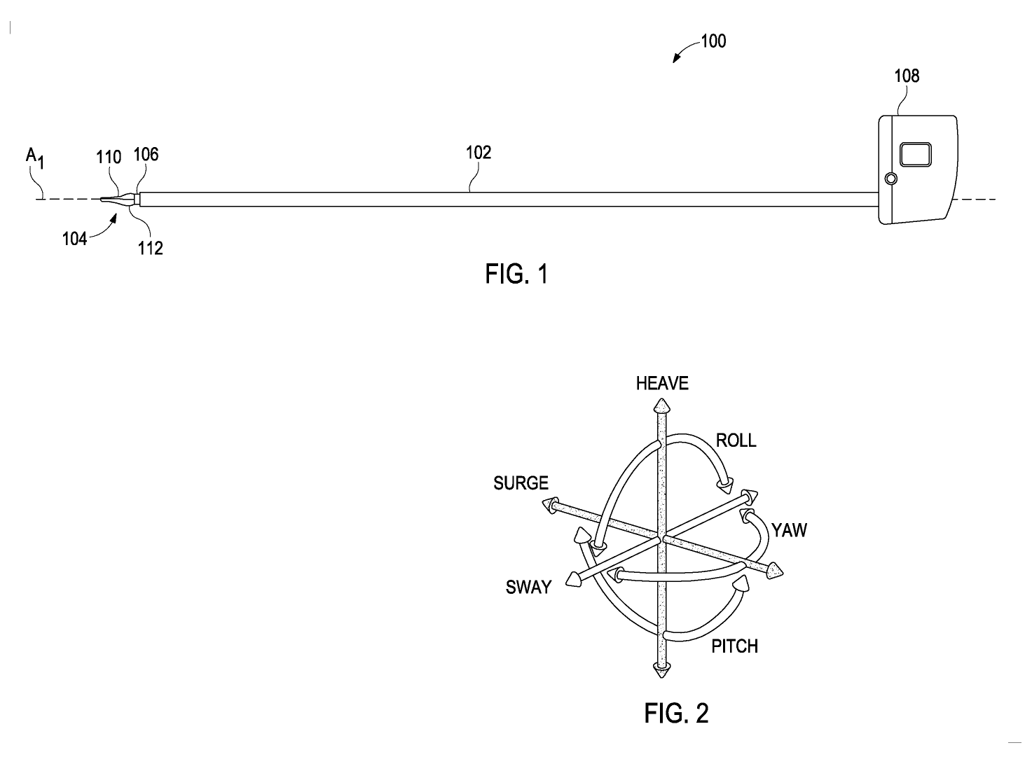

FIG. 1 is a side view of an example robotic surgical tool 100 that may incorporate some or all of the principles of the present disclosure. As illustrated, the robotic surgical tool 100 includes an elongate shaft 102, an end effector 104, a wrist 106 (alternately referred to as a “wrist joint”) that couples the end effector 104 to the distal end of the shaft 102, and a drive housing 108 coupled to the proximal end of the shaft 102. In at least some embodiments, the robotic surgical tool 100 may be designed to be releasably coupled to a robotic surgical system, and the drive housing 108 can include coupling features that releasably couple the robotic surgical tool 100 to the robotic surgical system.

The terms “proximal” and “distal” are defined herein relative to a robotic surgical system having an interface configured to mechanically and electrically couple the robotic surgical tool 100 (e.g., the housing 108) to a robotic manipulator. The term “proximal” refers to the position of an element closer to the robotic manipulator and the term “distal” refers to the position of an element closer to the end effector 104 and thus further away from the robotic manipulator. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the robotic surgical tool 100, the end effector 104 is configured to move (pivot) relative to the shaft 102 at the wrist 106 to position the end effector 104 at a desired orientation and location relative to a surgical site. The housing 108 includes various mechanisms designed to control operation of various features associated with the end effector 104 (e.g., clamping, firing, rotation, articulation, energy delivery, etc.). In at least some embodiments, the shaft 102, and hence the end effector 104 coupled thereto, is configured to rotate about a longitudinal axis A₁ of the shaft 102. In such embodiments, at least one of the mechanisms included in the housing 108 is configured to control rotational movement of the shaft 102 about the longitudinal axis A₁.

The robotic surgical tool 100 can have any of a variety of configurations capable of performing at least one surgical function. For example, the robotic surgical tool 100 may include, but is not limited to, forceps, a grasper, a needle driver, scissors, an electro cautery tool, a stapler, a clip applier, a suction tool, an irrigation tool, an imaging device (e.g., an endoscope or ultrasonic probe), or any combination thereof. In some embodiments, the robotic surgical tool 100 may be further configured to apply energy to tissue, such as radiofrequency (RF) energy.

The shaft 102 is an elongate member extending distally from the housing 108 and has at least one lumen extending therethrough along its axial length. In some embodiments, the shaft 102 may be fixed to the housing 108, but could alternatively be rotatably mounted to the housing 108 to allow the shaft 102 to rotate about the longitudinal axis A₁. In yet other embodiments, the shaft 102 may be releasably coupled to the housing 108, which may allow a single housing 108 to be adaptable to various shafts having different end effectors.

The end effector 104 can have a variety of sizes, shapes, and configurations. In the illustrated embodiment, the end effector 104 comprises a tissue grasper having opposing jaws 110, 112 configured to move between open and closed positions. One or both of the jaws 110, 112 may be configured to pivot at the wrist 106 to move the end effector 104 between the open and closed positions. In other embodiments, however, the end effector 104 may have other configurations, e.g., scissors including a pair of opposed cutting blades, a babcock including a pair of opposed grasping jaws, a retractor, a hook, a spatula, needle drivers, graspers, forceps, etc.

The wrist 106 can have any of a variety of configurations. In general, the wrist 106 comprises a joint configured to allow pivoting movement of the end effector 104 relative to the shaft 102.

FIG. 2 illustrates the potential degrees of freedom in which the wrist 106 may be able to articulate (pivot). The degrees of freedom of the wrist 106 are represented by three translational variables (i.e., surge, heave, and sway), and by three rotational variables (i.e., Euler angles or roll, pitch, and yaw). The translational and rotational variables describe the position and orientation of a component of a surgical system (e.g., the end effector 104) with respect to a given reference Cartesian frame. As depicted in FIG. 2, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 106 (e.g., X-axis), yaw movement about a second axis of the wrist 106 (e.g., Y-axis), and combinations thereof to allow for 360° rotational movement of the end effector 104 about the wrist 106. In other applications, the pivoting motion can be limited to movement in a single plane, e.g., only pitch movement about the first axis of the wrist 106 or only yaw movement about the second axis of the wrist 106, such that the end effector 104 moves only in a single plane.

Referring again to FIG. 1, the robotic surgical tool 100 includes a plurality of drive cables (obscured in FIG. 1) that form part of a cable driven motion system configured to effect movement (pivoting) of the end effector 104 relative to the shaft 102. The drive cables may be referred to and otherwise characterized as cables, bands, lines, cords, wires, ropes, strings, twisted strings, elongate members, etc. The drive cables can be made from a variety of materials including, but not limited to, metal (e.g., tungsten, stainless steel, etc.) or a polymer.

The drive cables are operably coupled to various actuation mechanisms housed within the drive housing 108 and extend within the lumen of the shaft 102 to the wrist 106 where they are operably engaged with the end effector 104. Selective actuation of all or a portion of the drive cables causes the end effector 104 (e.g., one or both of the jaws 110, 112) to move (pivot) relative to the shaft 102. More specifically, selective actuation causes a corresponding drive cable to translate longitudinally within the lumen of the shaft 102 and thereby cause pivoting movement of the end effector 104. In operation, one or more drive cables may translate longitudinally to cause the end effector 104 to articulate (e.g., both of the jaws 110, 112 angle in a same direction), to cause the end effector 104 to open (e.g., one or both of the jaws 110, 112 move away from the other), or to cause the end effector 104 to close (e.g., one or both of the jaws 110, 112 move toward the other).

Actuation of the drive cables can be accomplished in a variety of ways, such as by triggering an associated actuator operably coupled to or housed within the drive housing 108. Actuation applies tension to (i.e., pulls) the drive cables in a proximal direction to cause the corresponding elongate member to translate and thereby cause the end effector 104 to move (articulate) relative to the shaft 102. When both of the jaws 110, 112 are designed to move to open and close the end effector 104, one or more first drive cables will be operably coupled to the first jaw 110 to move that jaw 110 and one or more second drive cables will be operably coupled to the second jaw 112 to move that jaw 112. When only one of the jaws 110, 112 is configured to move to open and close the end effector 104, one or more drive cables may be operably coupled to the first jaw 110 to move the first jaw 110 relative to the second jaw 112.

Actuating the drive cables moves the end effector 104 between an unarticulated position and an articulated position. The end effector 104 is depicted in FIG. 1 in the unarticulated position where a longitudinal axis A₂ of the end effector 104 is substantially aligned with the longitudinal axis A₁ of the shaft 102, such that the end effector 104 is at a substantially zero angle relative to the shaft 102. Due factors such as manufacturing tolerance and precision of measurement devices, the end effector 104 may not be at a precise zero angle relative to the shaft 102 in the unarticulated position, but nevertheless be considered “substantially aligned” thereto. In the articulated position, the longitudinal axes A₁, A₂ would be angularly offset from each other such that the end effector 104 is at a non-zero angle relative to the shaft 102.

The drive housing 108 (alternately referred to as a “puck”) may be releasably latched (attached) to a tool driver of a robotic surgical system in a variety of ways, such as by clamping thereto, clipping thereto, or slidably mating therewith. The actuation devices or mechanisms housed within the drive housing 108 may be controlled by the robot based on user inputs received via a computer system incorporated into the robot. Accordingly, the user inputs control movement of the drive cables and consequently movement of the end effector 104.

Example tool drivers to which the drive housing 108 may be removably attached are described in U.S. Patent Application No. 15/200,283, titled “Methods, systems, and devices for initializing a surgical tool”, which issued as U.S. Pat. No. 10,149,726, which is hereby incorporated by reference in its entirety. Moreover, the drive housing 108 illustrated in FIG. 1 is but one example of a suitable drive housing, and additional embodiments of the drive housing 108 are described in U.S. Patent Pub. No. 2015/0209965, titled “Compact robotic wrist”, which issued as U.S. Pat. No. 9,884,427, and U.S. Pub. No. 2015/0025549, titled “Hyperdexterous surgical system”, which published on Jan. 22, 2015, both of which are incorporated by reference in their entireties. Example robotic surgical systems are described in U.S. Patent No. 8,831,782, titled “Patient-Side Surgeon Interface for a Teleoperated Surgical Instrument”, which issued on Sep. 9, 2014, which is hereby incorporated by reference in its entirety, as well as previously mentioned U.S. Patent Pub. Nos. 2015/0209965 and 2015/0025549.

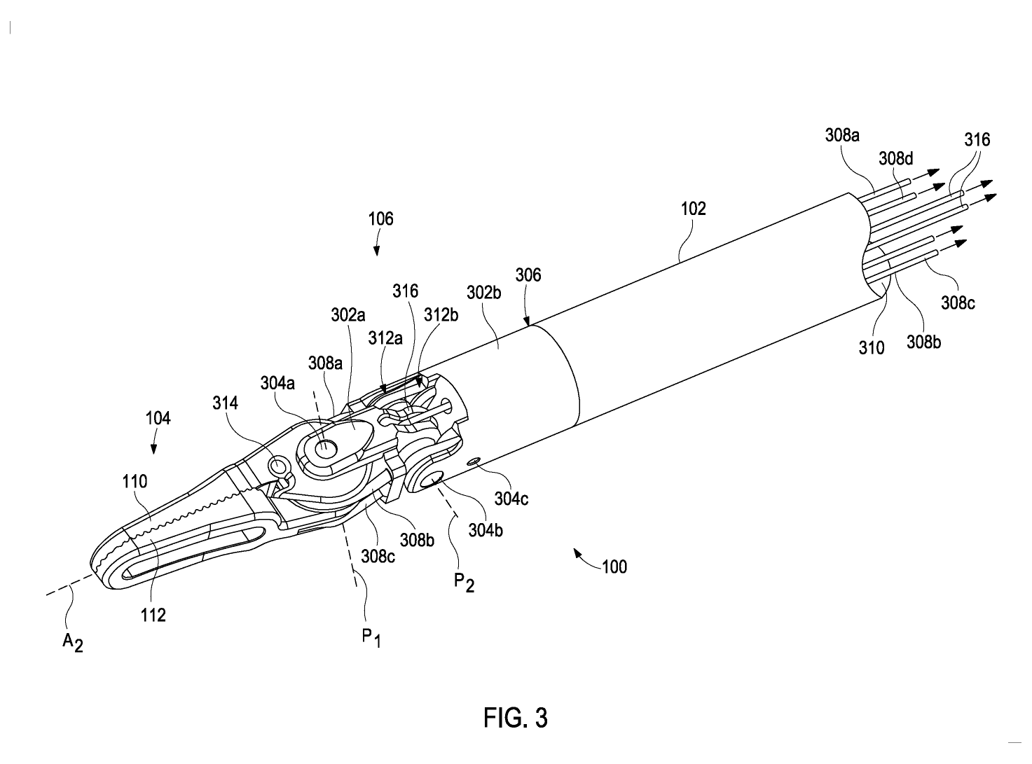

FIG. 3 is an enlarged isometric view of the distal end of the robotic surgical tool 100 of FIG. 1. More specifically, FIG. 3 depicts enlarged views of the end effector 104 and the wrist 106, with the end effector 104 in the unarticulated position where the jaws 110, 112 are closed (in a closed configuration). The wrist 106 operatively couples the end effector 104 to the shaft 102. To accomplish this, the wrist 106 includes a distal clevis 302a and a proximal clevis 302b. The end effector 104 (i.e., the jaws 110, 112) is rotatably mounted to the distal clevis 302a at a first axle 304a, the distal clevis 302a is rotatably mounted to the proximal clevis 302b at a second axle 304b, and the proximal clevis 302b is coupled to a distal end 306 of the shaft 102.

The wrist 106 provides a first pivot axis P₁ that extends through the first axle 304a and a second pivot axis P₂ that extends through the second axle 304b. The first pivot axis P₁ is substantially perpendicular (orthogonal) to the longitudinal axis A₂ of the end effector 104, and the second pivot axis P₂ is substantially perpendicular to both the longitudinal axis A₂ and the first pivot axis P₁. Movement about the first pivot axis P₁ provides “yaw” articulation of the end effector 104, and movement about the second pivot axis P₂ provides “pitch” articulation of the end effector 104. In the illustrated embodiment, the jaws 110, 112 are mounted at the first pivot axis P₁, thereby allowing the jaws 110, 112 to pivot relative to each other to open and close the end effector 104 or alternatively pivot in tandem to articulate the orientation of the end effector 104.

A plurality of drive cables 308, shown as drive cables 308a, 308b, 308c, and 308d, extend longitudinally within a lumen 310 of the shaft 102 until terminating at the wrist 106. The drive cables 308a-d extend proximally from the end effector 104 to the drive housing 108 (FIG. 1) which, as discussed above, may be configured to facilitate longitudinal movement of the drive cables 308a-d within the lumen 310. The lumen 310 can be a single lumen, as illustrated, or can alternatively comprise a plurality of independent lumens that each receive one or more of the drive cables 308a-d.

The wrist 106 includes a first plurality of pulleys 312a and a second plurality of pulleys 312b each configured to interact with and redirect the drive cables 308a-d for engagement with the end effector 104. The first plurality of pulleys 312a is mounted to the proximal clevis 302b at the second axle 304b and the second plurality of pulleys 312b is mounted to the proximal clevis 302b at a third axle 304c. The third axle 304c is located proximal to the second axle 304b. The first and second plurality of pulleys 312a,b cooperatively redirect the drive cables 308a-d through an “S” shaped pathway.

In at least one embodiment, one pair of drive cables 308a-d is operatively coupled to each jaw 110, 112 and configured to “antagonistically” operate the corresponding jaw 110, 112. In the illustrated embodiment, for example, the first and second drive cables 308a,b are coupled at a connector 314 mounted to the first jaw 110, and the third and fourth drive cables 308c,d are coupled at another connector (hidden in FIG. 3) mounted to the second jaw 112. Actuation of the first drive cable 308a acts on the connector 314 and thereby pivots the first jaw 110 about the first pivot axis P₁ toward the open position. In contrast, actuation of the second drive cable 308b acts on the connector 314 and thereby pivots the first jaw 110 about the first pivot axis P₁ in the opposite direction and toward the closed position. Similarly, actuation of the third drive cable 308c acts on the corresponding connector (not shown) and thereby pivots the second jaw 112 about the first pivot axis P₁ toward the open position, while actuation of the fourth drive cable 308d acts on the corresponding connector to pivot the second jaw 112 about the first pivot axis P₁ in the opposite direction and toward the closed position.

Accordingly, the drive cables 308a-d may be characterized or otherwise referred to as “antagonistic” cables that cooperatively (antagonistically) operate to cause relative or tandem movement of the first and second jaws 110, 112. When the first drive cable 308a is actuated, the second drive cable 308b naturally follows as coupled to the first drive cable 308a at the connector 314, and vice versa. Similarly, when the third drive cable 308c is actuated, the fourth drive cable 308d naturally follows as coupled to the third drive cable 308c at the other connector (hidden in FIG. 3), and vice versa.

Moreover, coordinated actuation of the drive cables 308a-d may also articulate the end effector 104 about the second pivot axis P₂. Consequently, the end effector 104 can articulate with multiple degrees of freedom, e.g., a degree of freedom by articulating about the first pivot axis P₁ and another degree of freedom by articulating about the second pivot axis P₂. The wrist 106 in this embodiment is pivotable about the second pivot axis P₂ in a single plane, e.g., in one of pitch and yaw, and the end effector 104 is pivotable about the first pivot axis P₁ in a single, different plane, e.g., the other of pitch and yaw.

Additional information regarding the robotic surgical tool 100 is provided in U.S. Patent No. 10,973,600, issued April 13, 2021 and titled “POWER AXLE WRIST FOR ROBOTIC SURGICAL TOOL”, the disclosure of which is hereby incorporated by reference in its entirety herein.

During a surgical procedure, the end effector 104 of the robotic surgical tool 100 is commonly used to grasp and manipulate the tissue of a patient. While grasping and manipulating the tissue, there is a chance that the tissue may slip relative to the end effector 104. One possible solution may be to increase the grip force applied to the tissue by the end effector 104. However, the amount of grip force that the jaws 110, 112 of the end effector 104 jaws can apply to tissue is significantly less compared to the amount of grip force that the jaws of an end effector of a handheld surgical tool can apply to tissue. Furthermore, increasing the grip force may shorten the life of the robotic surgical tool 100. For example, increasing the grip force may require providing an increased tensioning force on the drive cables, which may cause the drive cables to eventually strain and fail. Accordingly, an improved end effector is desired to improve tissue grip without increasing grip strength.

### Improvements to Grasper Designs

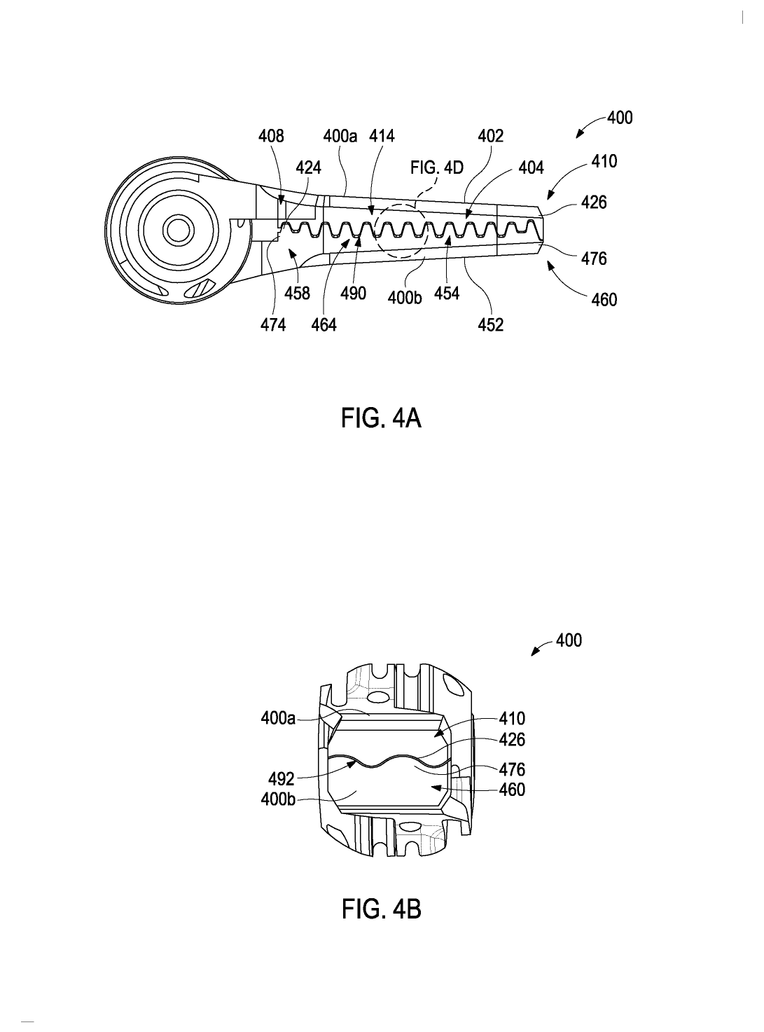

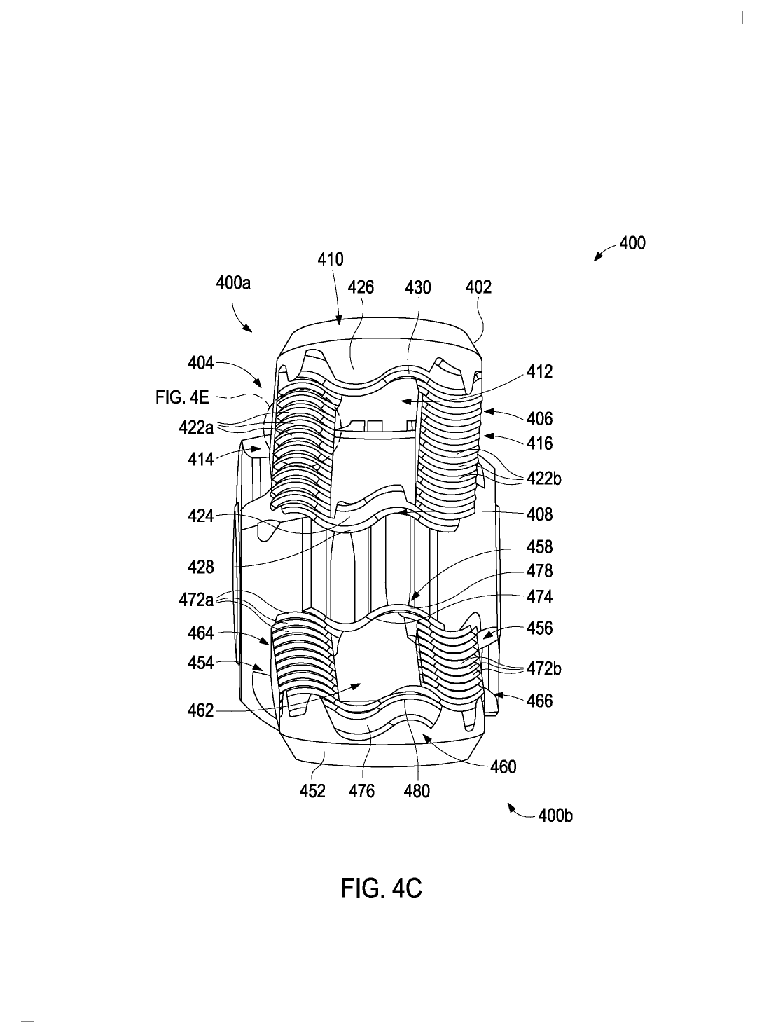

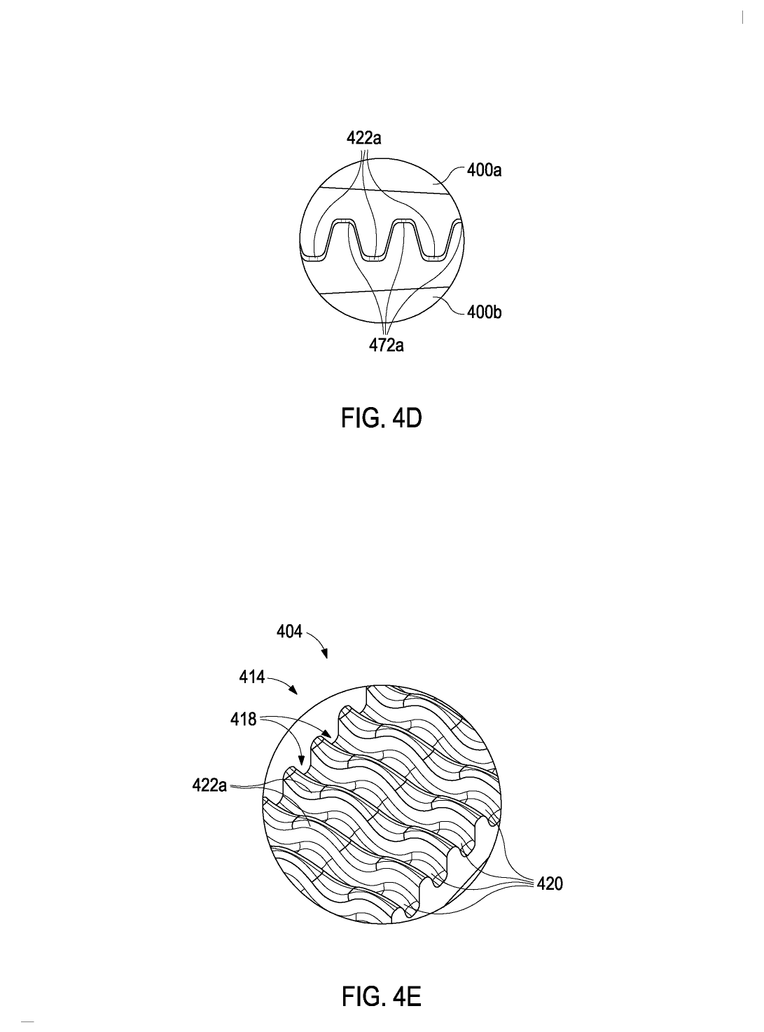

FIGS. 4A-4E are enlarged views of first and second jaws 400a, 400b of an example end effector 400, in accordance with at least one aspect of the present disclosure. The end effector 400, the first jaw 400a, and the second jaw 400b may be similar to or the same as the end effector 104, the first jaw 110, and the second jaw 112, respectively, of FIG. 1 and therefore may be best understood with reference thereto. Accordingly, in at least one application, the end effector 400 may replace the end effector 104 (FIG. 1) for the robotic surgical tool 100 (FIG. 1).

With particular reference to FIG. 4C, the first jaw 400a may include a first jaw body 402 that includes a first lateral portion 404 on a first lateral side of the end effector 400, a second lateral portion 406 on a second lateral side of the end effector 400 opposite the first lateral side, a proximal portion 408 arranged proximal to and extending between the first and second lateral sides of the end effector 404, and a distal portion 410 arranged distal to and extending between the first and second lateral sides of the end effector 404. The first and second lateral portions 404, 406, the proximal portion 408, and the distal portion 410 may co-operatively define an aperture 412 (alternately referred to as a “fenestration”) entirely through the first jaw 400a. The aperture 412 separates the first and second lateral portions 404, 406 and the proximal and distal portions 408, 410. In alternative embodiments, the first jaw 400a may not include the aperture 412. Rather, in such embodiments, the first jaw body 402 may include a central structural portion (not shown) arranged between the first and second lateral portions 404, 406 and the proximal and distal portions 408, 410 to occlude the aperture 412.

The first jaw 400a may further include first extensions or “grips” extending from the first jaw body 402 toward the second jaw 400b. The first grips may include first lateral grips 414 extending from the first lateral portion 404 of the first jaw body 402. The first lateral grips 414 may comprise a series of grip members or ridges longitudinally spaced from one another along the length of the first lateral portion 404 to define gaps 418 (see FIG. 4E) therebetween. The first lateral grips 414 may have a width that spans, or at least substantially spans, the width of the first lateral portion 404.

With reference to FIG. 4E, the first lateral portion 404 may provide tissue contacting surfaces 420 that vertically undulate toward and away from the second jaw 400b along the width of the first lateral portion 404 (e.g., are vertically non-linear and/or extend laterally non-linearly). The tissue contacting surfaces 420 may be arranged (located) in the gaps 418 between the longitudinally spaced first lateral grips 414. Furthermore, the first lateral grips 414 may provide tissue contacting surfaces 422a that vertically undulate toward and away from the second jaw 400b along the width of the first lateral grips 414 (e.g., are vertically non-linear and/or extend laterally non-linearly). As shown in FIG. 4E, the vertically undulating profile of the tissue contacting surfaces 420, 422a may be identical along the width of the first lateral portion 404 (i.e., the crests and peaks of the tissue contacting surfaces 420, 422a may longitudinally align). In other embodiments, the undulating profile of the tissue contacting surfaces 420, 422a may be different along the width of the first lateral portion 404 (i.e., the crests and peaks of the tissue contacting surfaces 420, 422a may not longitudinally align).

Referring again to FIG. 4C, the first grips may include second lateral grips 416 extending from the second lateral portion 406 of the first jaw body 402. The second lateral grips 416 may comprise a series of grip members or ridges longitudinally spaced from one another along the length of the second lateral portion 406 to define gaps (similar to gaps 418; FIG. 4E) therebetween. The second lateral grips 416 may have a width that spans, or at least substantially spans, the width of the second lateral portion 406.

The second lateral portion 406 may provide tissue contacting surfaces similar to tissue contacting surfaces 420 of FIG. 4E that vertically undulate toward and away from the second jaw 400b along the width of the second lateral portion 406 (e.g., are vertically non-linear and/or extend laterally non-linearly). The tissue contacting surfaces may be arranged (located) in the gaps between the longitudinally spaced second lateral grips 416, similar to what is shown in FIG. 4E. Furthermore, the second lateral grips 416 may provide tissue contacting surfaces 422b that vertically undulate toward and away from the second jaw 400b along the width of the second lateral grips 416 (e.g., are vertically non-linear and/or extend laterally non-linearly). Similar to the first lateral portion 404, the vertically undulating profile of the tissue contacting surfaces of the second lateral portion 406 may be identical along the width of the second lateral portion 406 (i.e., the crests and peaks of the tissue contacting surfaces may longitudinally align). In other embodiments, the undulating profile of the tissue contacting surfaces of the second lateral portion 406 may be different along the width of the second lateral portion 406 (i.e., the crests and peaks of the tissue contacting surfaces may not longitudinally align).

The first grips may further include a proximal grip 424 extending from the proximal portion 408 of the first jaw 400a toward the second jaw 400b and a distal grip 426 extending from the distal portion 410 of the first jaw 400a toward the second jaw 400b. The proximal and distal grips 424, 426 may have a width that spans, or at least substantially spans, the width of the proximal and distal portions 408, 410 of the first jaw 400a, respectively. The proximal and distal grips 424, 426 exhibit a width greater than the width of the first and second lateral grips 414, 416. Moreover, the proximal grip 424 may include a proximal tissue contacting surface 428 that vertically undulates toward and away from the second jaw 400b (e.g. is vertically non-linear) along the width of the proximal portion 408 of the first jaw 400a. Similarly, the distal grip 426 may include a distal tissue contacting surface 430 that vertically undulates toward and away from the second jaw 400b (e.g. is vertically non-linear) along the width of the distal portion 410 of the first jaw 400a.

Similar to the first jaw 400a, the second jaw 400b may include a second jaw body 452 that includes a first lateral portion 454 on the first lateral side of the end effector 400, a second lateral portion 456 on the second lateral side of the end effector 400, a proximal portion 458 arranged proximal to and extending between the first and second lateral sides of the second jaw 400b, and a distal portion 460 arranged distal to and extending between the first and second lateral sides of the end effector 404. In some embodiments, the first and second lateral portions 454, 456, the proximal portion 458, and the distal portion 460 may co-operatively form an aperture 462 defined entirely through the second jaw 400b. The aperture 462 separates the first lateral portion 454 from the second lateral portion 456, and the proximal portion 458 from the distal portion 460. When the first and second jaw 400a, 400b are in the closed configuration, as shown in FIGS. 4A and 4B, the first and second lateral portions 454, 456, the proximal portion 458, and the distal portion 460 of the second jaw 400b may oppose (confront) the first and second lateral portions 404, 406, the proximal portion 408, and the distal portion 410 of the first jaw 400a, respectively. In alternative embodiments, the second jaw 400b may not define the aperture 462. Rather, in such embodiments, the second jaw body 452 may include a central structural portion (not shown) arranged between the first and second lateral portions 454, 456 and the proximal and distal portions 458, 460 to occlude the aperture 462.

The second jaw 400b may further include second extensions or “grips” extending from the second jaw body 452 toward the first jaw 400a. The second grips may include first lateral grips 464 extending from the first lateral portion 454 of the second jaw body 452. The first lateral grips 464 may comprise a series of grip members or ridges longitudinally spaced from one another along the length of the second jaw body 452 to define gaps (similar to gaps 418) therebetween. The first lateral grips 464 may have a width that spans, or at least substantially spans, the width of the first lateral portion 454.

The first lateral portion 454 of the second jaw 400b may provide tissue contacting surfaces (not visible) similar to tissue contacting surfaces 420 of FIG. 4E that vertically undulate toward and away from the first jaw 400a along the width of the first lateral portion 454 (e.g., are vertically non-linear and/or extend laterally non-linearly). The tissue contacting surfaces may be arranged (located) in the gaps between the longitudinally spaced first lateral grips 464, similar to what is shown in FIG. 4E. Furthermore, the first lateral grips 464 may provide tissue contacting surfaces 472a that vertically undulate toward and away from the first jaw 400a along the width of the first lateral grips 464 (e.g., are vertically non-linear and/or extend laterally non-linearly). Similar what is shown in FIG. 4E, the vertically undulating profile of the tissue contacting surfaces may be identical along the width of the first lateral portion 454 (i.e., the crests and peaks of the tissue contacting surfaces may longitudinally align). In other embodiments, the undulating profile of the tissue contacting surfaces may be different along the width of the first lateral portion 454 (i.e., the crests and peaks of the tissue contacting surfaces may not longitudinally align).

Similarly, the second grips may include second lateral grips 466 extending from the second lateral portion 456 of the second jaw body 452. The second lateral grips 466 may comprise grip members or ridges longitudinally spaced from one another along the length of the second jaw body 452 to define gaps (similar to gaps 418) therebetween. The second lateral grips 466 may have a width that spans, or at least substantially spans, the width of the second lateral portion 456.

The second lateral portion 456 of the second jaw 400b may provide tissue contacting surfaces (not visible) similar to tissue contacting surfaces 420 of FIG. 4E that vertically undulate toward and away from the first jaw 400a along the width of the second lateral portion 456 (e.g., are vertically non-linear and/or extend laterally non-linearly). The tissue contacting surfaces may be arranged (located) in the gaps between the longitudinally spaced apart second lateral grips 466, similar to what is shown in FIG. 4E. Furthermore, the second lateral grips 466 may provide tissue contacting surfaces 472b that vertically undulate toward and away from the first jaw 400a along the width of the second lateral grips 466 (e.g., are vertically non-linear and/or extend laterally non-linearly). Similar what is shown in FIG. 4E, the vertically undulating profile of the tissue contacting surfaces of the second lateral portion 456 may be identical along the width of the second lateral portion 456 (i.e., the crests and peaks of the tissue contacting surfaces may longitudinally align). In other embodiments, the undulating profile of the tissue contacting surfaces of the second lateral portion 456 may be different along the width of the second lateral portion 456 (i.e., the crests and peaks of the tissue contacting surfaces may not longitudinally align).

The second grips may further include a proximal grip 474 extending from the proximal portion 458 of the second jaw 400b toward the first jaw 400a, and a distal grip 476 extending from the distal portion 460 of the second jaw 400b toward the first jaw 400a. The proximal and distal grip 474, 476 exhibit a width that spans, or at least substantially spans, the width of the proximal and distal portion 458, 460 of the second jaw 400b, respectively. The proximal and distal grips 474, 476 may have a width that is greater than the width of the first and second lateral grips 464, 466. The proximal grip 474 may include a proximal tissue contacting surface 478 that vertically undulates toward and away from the first jaw 400a (e.g. is vertically non-linear) along the width of the proximal portion 458 of the second jaw 400b. Similarly, the distal grip 476 may include a distal tissue contacting surface 480 that vertically undulates toward and away from the first jaw 400a (e.g. is vertically non-linear) along the width of the distal portion 460 of the second jaw 400b.

As described above, the first and second lateral grips 414, 416, 464, 466 may be longitudinally spaced apart along the lengths of the first and second jaw bodies 402, 452, respectively, to define gaps (like gaps 418; FIG. 4E) therebetween. Moreover, the first lateral grips 414, 464 may be longitudinally offset from one another such that, when the first and second jaws 400a, 400b are transitioned from an open configuration, as shown in FIG. 4C, to a closed configuration, as shown in FIGS. 4A and 4B, the first lateral grips 414 of the first jaw 400a mesh with the first lateral grips 464 of the second jaw 400b in a nested relationship. More specifically, when the first and second jaws 400a, 400b close, the tissue contacting surfaces 422a of the first lateral grips 414 are received within the gaps defined between the first lateral grips 464 and oppose (confront) the tissue contacting surfaces of the first lateral portion 454, and the tissue contacting surfaces 472a of the first lateral grips 464 are received within the gaps 418 defined between the first lateral grips 414 and oppose (confront) the tissue contacting surfaces 420 of the first lateral portion 404. This mated and nested relationship defines a first non-linear longitudinal interface 490, as shown in FIG. 4A, along the longitudinal length of the first and second jaws 400a, 400b on the first lateral side of the end effector 400.

The vertically undulating profile of the tissue contact surfaces 422a may be complimentary to (the same, or substantially the same, as) the vertically undulating profile of the tissue contacting surfaces of the first lateral portion 454. Similarly, the vertically undulating profile of the tissue contact surfaces 472a may be complimentary to (the same, or substantially the same, as) the vertically undulating profile of the tissue contacting surfaces 420 of the first lateral portion 404.

When the first and second jaws 400a, 400b are closed, the first non-linear longitudinal interface 490 creates no (or substantially no) line of sight gap that would otherwise be visible laterally between the first lateral portions 404, 464. For instance, when the first lateral grips 414, 464 are meshed, as described above, a direct, linear line of sight through the jaws 400a,b is not provided between the first lateral portions 404, 464.

Similarly, the second lateral grips 416, 466 longitudinally offset one another such that, when the first and second jaws 400a, 400b are closed, the second lateral grips 416 of the first jaw 400a mesh with the second lateral grips 466 of the second jaw 400b in a nested relationship. More specifically, when the first and second jaws 400a, 400b are closed, the tissue contacting surfaces 422b of the second lateral grips 416 are received within the gaps between the second lateral grips 466 and oppose (confront) the tissue contacting surfaces of the second lateral portion 456, and the tissue contacting surfaces 472b of the second lateral grips 466 received within the gaps between the second lateral grips 416 and oppose (confront) the tissue contacting surfaces of the second lateral portion 406. This mated and nested relationship defines a second non-linear longitudinal interface similar to the first non-linear longitudinal interface 490 along the length of the first and second jaws 400a, 400b, but on the second lateral side of the end effector 400.

The vertically undulating profile of the tissue contact surfaces 422b may be complimentary to (the same, or substantially the same, as) the vertically undulating profile of the tissue contacting surfaces of the second lateral portion 456. Similarly, the vertically undulating profile of the tissue contact surfaces 472b may be complimentary to (the same, or substantially the same, as) the vertically undulating profile of the tissue contacting surfaces of the second lateral portion 406.

When the first and second jaws 400a, 400b are closed, the second non-linear longitudinal interface creates no (or substantially no) line of sight gap that would otherwise be visible laterally between the second lateral portions 406, 466. For instance, when the second lateral grips 416, 466 are meshed, as described above, a direct, linear line of sight through the jaws 400a,b is not provided between the second lateral portions 406, 466.

Furthermore, when the first and second jaws 400a, 400b are placed in the closed configuration, the distal tissue contacting surface 430 of the first jaw 400a may confront (mesh with) the distal tissue contacting surface 480 of the second jaw 400b, thereby defining a first non-linear lateral interface 492 (FIG. 4B) along the width of the first and second jaws 400a, 400b at a distal end the end effector 400. The vertically undulating profile of the tissue contact surface 430 may be the same, or substantially the same, as the vertically undulating profile of the tissue contacting surface 480.

Similarly, when the first and second jaws 400a, 400b are closed, the proximal tissue contacting surface 428 of the first jaw 400a may confront (mesh with) the proximal tissue contacting surface 478 of the second jaw 400b, thereby defining a second non-linear lateral interface (not shown, but similar to the first non-linear lateral interface 492) along the width of the first and second jaws 400a, 400b at a proximal end the end effector 400. The vertically undulating profile of the tissue contact surface 428 may be the same, or substantially the same, as the vertically undulating profile of the tissue contacting surface 478.

The first, non-linear lateral interface 492, the opposing second non-linear lateral interface, the first non-linear longitudinal interface 490, and the opposing second non-linear longitudinal interface may collectively (cooperatively) define a substantially non-linear interface between the first and second jaws 400a, 400b that spans the perimeter of the end effector 400 when the first and second jaws 400a, 400b are in the closed configuration, which may obscure (prevent), a direct, linear line of sight between the jaws 404a,b from any direction.

The end effector 400 provides numerous benefits over traditional end effectors, as will be readily apparently to those skilled in the art. For example, the grips 414, 416, 464, 466 provide an increased, substantially “slip free” grip on tissue, as compared to traditional end effectors, without requiring an increased grip force. Accordingly, the end effector 400 may be particularly beneficial due to the reduced amount of grip force that may be applied by robotic surgical instruments. Moreover, the end effector 400 may be applied to various other surgical instruments, such as handheld surgical instruments.

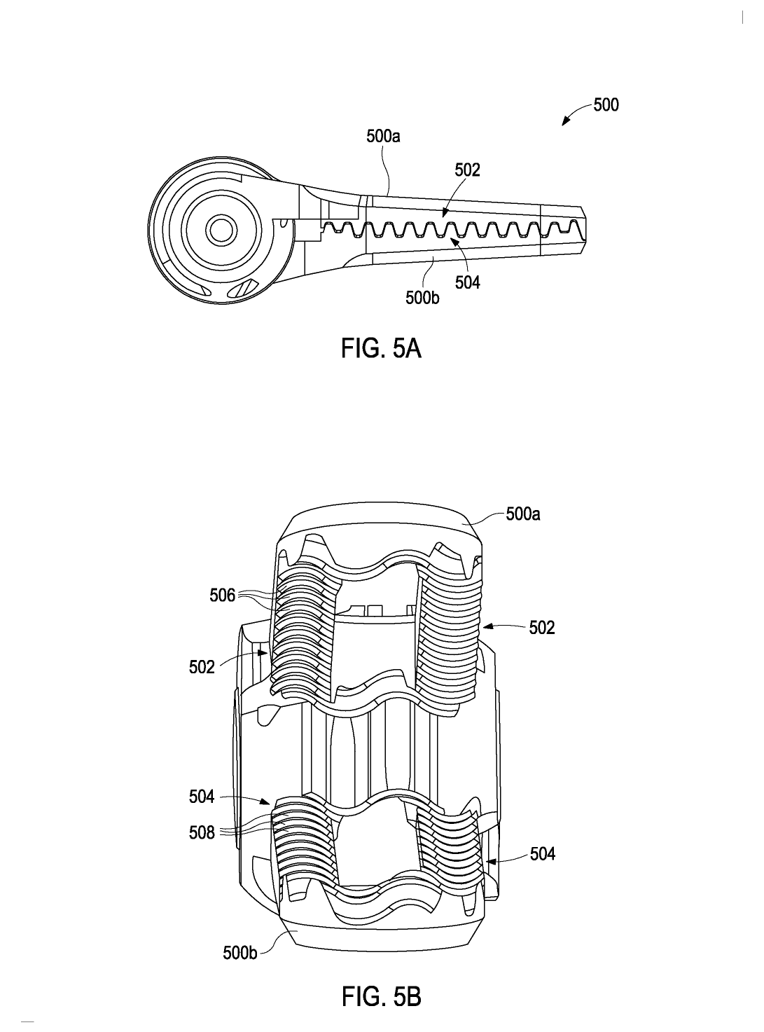

FIGS. 5A and 5B are enlarged views of first and second jaws 500a, 500b of another example end effector 500, in accordance with one or more additional aspects of the present disclosure. The end effector 500 may be similar in some respects to the end effector 400 (FIGS. 4A-4E) and/or the end effector 104 (FIG. 1), and therefore may be best understood with reference thereto. In at least one application, the end effector 500 may replace the end effector 104 in the robotic surgical tool 100 (FIG. 1).

Similar to the end effectors 104, 400, the opposing first and second jaws 500a,b may be configurable between a closed configuration, as shown in FIG. 5A, and an open configuration, as shown in FIG. 5B. A first grip 502 may be provided or otherwise formed on the first jaw 500a, and may be substantially similar to the first grips of the first jaw 400a (FIGS. 4A-4E). Similarly, a second grip 504 may be provided or otherwise formed on the second jaw 500b, and may be substantially similar to the second grips of the second jaw 400b (FIGS. 4A-4E). As seen best in FIG. 5B, the first grip 502 may define a plurality of undulating and non-linear grooves 506 along the width of the first jaw 500a. Similarly, the second grip 504 may define a plurality of undulating and non-linear grooves 508 along the width of the second jaw 500b.

Referring particularly to FIG. 5A, when the first and second jaws 110, 112 are closed (e.g., placed in the closed configuration), the grips 502, 504 and corresponding grooves 506, 508 mate such that no line of sight may be visible laterally through the first and second jaws 110, 112. For instance, due to the laterally undulating and non-linear grips 502, 504 and grooves 506, 508, a direct, linear line of sight is not provided between the first and second jaws 110, 112. Rather, the undulating and non-linear grooves 506, 508 prevent light, for example, from directly (linearly) passing laterally through the first and second jaws 110, 112.

### Embodiments disclosed herein include:

A. An end effector of a surgical tool, the end effector comprising a first jaw including a first jaw body and first grips extending from the first jaw body and a second jaw including a second jaw body and second grips extending from the second jaw body, the first and second jaws being movable between open and closed configurations, wherein the first and second grips mesh upon transitioning the first and second jaws to the closed configuration to thereby define a non-linear longitudinal interface along a length of the first and second jaws and a non-linear lateral interface along a width of the first and second jaws.

B. An end effector of a surgical tool, the end effector comprising opposing first and second jaws, a first grip formed on the first jaw and defining a first plurality of undulating and non-linear grooves, a second grip formed on the second jaw and defining a second plurality of undulating and non-linear grooves, wherein, when the first and second jaws close, the first and second pluralities of undulating and non-linear grooves mate such that no line of sight is visible laterally through the first and second jaws.

C. An end effector of a surgical tool, the end effector comprising a first jaw including first grips and a second jaw including second grips, the first and second jaws being transitionable between an open configuration and a closed configuration, wherein, when the first and second jaws are in the closed configuration, the first and second grips mesh to define a first non-linear longitudinal interface along a first lateral side of the end effector.

Each of embodiments A, B, and C may have one or more of the following additional elements in any combination: Element 1: wherein each first grip includes a first tissue contacting surface extending laterally and non-linearly, and each second grip includes a second tissue contacting surface extending laterally and non-linearly. Element 2: wherein the first grips include a first distal grip, the second grips include a second distal grip, and the first distal grip meshes with the second distal grip to define the non-linear lateral interface. Element 3: wherein the non-linear lateral interface is a first non-linear lateral interface, the first grips further include a first proximal grip, the second grips further include a second proximal grip, and the first proximal grip meshes with the second proximal grip to define a second non-linear lateral interface along the width of the first and second jaws. Element 4: wherein the first grips include first lateral grips extending from a first lateral portion of the first jaw body and the second grips include first lateral grips extending from a first lateral portion of the second jaw body, and wherein the first lateral grips of the first jaw mesh with the first lateral grips of the second jaw to define the non-linear longitudinal interface. Element 5: wherein the first grips further include second lateral grips extending from a second lateral portion of the first jaw body, and the second grips further include second lateral grips extending from a second lateral portion of the second jaw body, and wherein the second lateral grips of the first jaw mesh with the second lateral grips of the second jaw to define a second non-linear longitudinal interface. Element 6: wherein the first jaw defines an aperture that separates the first and second lateral portions of the first jaw. Element 7: wherein, when the first and second jaws close, a non-linear longitudinal interface is defined along a length of the first and second jaws. Element 8: wherein, when the first and second jaws close, a non-linear lateral interface is defined along a width of the first and second jaws. Element 9: wherein the first grips include first lateral grips longitudinally spaced apart to define first gaps therebetween and the second grips include first lateral grips longitudinally spaced apart to define second gaps therebetween, wherein, when in the closed configuration, the first lateral grips of the first jaw are received in the second gaps, and the first lateral grips of the second jaw are received in the first gaps to define the first non-linear longitudinal interface. Element 10: wherein the first lateral grips of the first jaw each include a tissue contacting surface that vertically undulates toward and away from the second jaw and the first lateral grips of the second jaw each include a tissue contacting surface that vertically undulates toward and away from the first jaw. Element 11: wherein the first jaw includes a first lateral portion and a second lateral portion, the first grips extend from the first lateral portion of the first jaw, the second jaw includes a first lateral portion and a second lateral portion, and the second grips extend from the first lateral portion of the second jaw. Element 12: wherein a first aperture is defined in the first jaw between the first and second lateral portions of the first jaw and a second aperture is defined in the second jaw between the first and second lateral portions of the second jaw. Element 13: wherein the first grips further include second lateral grips longitudinally spaced apart along the second lateral portion of the first jaw to define third gaps therebetween and the second grips further include second lateral grips longitudinally spaced apart along the second lateral portion of the second jaw to define fourth gaps therebetween, wherein, when in the closed configuration, the second lateral grips of the first jaw are received in the fourth gaps and the second lateral grips of the second jaw are received in the third gaps to define a second non-linear longitudinal interface along a second lateral side of the end effector. Element 14: wherein the second lateral grips of the first jaw each include a tissue contacting surface that vertically undulates toward and away from the second jaw and the second lateral grips of the second jaw each include a tissue contacting surface that vertically undulates toward and away from the first jaw. Element 15: wherein the first grips include a first distal grip, the second grips include a second distal grip and the first distal grip meshes with the second distal grip to define a first non-linear lateral interface along the width of the first and second jaws. Element 16: wherein the first grips further include a first proximal grip, the second grips further include a second proximal grip, and the first proximal grip meshes with the second proximal grip to define a second non-linear lateral interface along the width of the first and second jaws.

By way of non-limiting example, exemplary combinations applicable to A, B, and C include: Element 2 with Element 3; Element 4 with Element 5; Element 4 with Elements 5 and 6; Element 9 with Element 10; Element 9 with Element 11; Element 9 with Elements 11 and 12; Element 9 with Elements 11 and 13; Element 9 with Elements 11, 13, and 14; Element 15 with Element 16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. An end effector of a surgical tool, the end effector comprising:
a first jaw including a first jaw body and first grips extending from the first jaw body; and
a second jaw including a second jaw body and second grips extending from the second jaw body, the first and second jaws being movable between open and closed configurations,
wherein the first and second grips mesh upon transitioning the first and second jaws to the closed configuration to thereby define:
a non-linear longitudinal interface along a length of the first and second jaws; and
a non-linear lateral interface along a width of the first and second jaws.

2. The end effector of claim 1, wherein each first grip includes a first tissue contacting surface extending laterally and non-linearly, and each second grip includes a second tissue contacting surface extending laterally and non-linearly.

3. The end effector of claim 1, wherein:
the first grips include a first distal grip;
the second grips include a second distal grip; and
the first distal grip meshes with the second distal grip to define the non-linear lateral interface.

4. The end effector of claim 3, wherein:
the non-linear lateral interface is a first non-linear lateral interface;
the first grips further include a first proximal grip;
the second grips further include a second proximal grip; and
the first proximal grip meshes with the second proximal grip to define a second non-linear lateral interface along the width of the first and second jaws.

5. The end effector of claim 1, wherein the first grips include first lateral grips extending from a first lateral portion of the first jaw body and the second grips include first lateral grips extending from a first lateral portion of the second jaw body, and wherein the first lateral grips of the first jaw mesh with the first lateral grips of the second jaw to define the non-linear longitudinal interface.

6. The end effector of claim 5, wherein the first grips further include second lateral grips extending from a second lateral portion of the first jaw body, and the second grips further include second lateral grips extending from a second lateral portion of the second jaw body, and wherein the second lateral grips of the first jaw mesh with the second lateral grips of the second jaw to define a second non-linear longitudinal interface.

7. The end effector of claim 6, wherein the first jaw defines an aperture that separates the first and second lateral portions of the first jaw.

8. An end effector of a surgical tool, the end effector comprising:
opposing first and second jaws;
a first grip formed on the first jaw and defining a first plurality of undulating and non-linear grooves;
a second grip formed on the second jaw and defining a second plurality of undulating and non-linear grooves,
wherein, when the first and second jaws close, the first and second pluralities of undulating and non-linear grooves mate such that no line of sight is visible laterally through the first and second jaws.

9. The end effector of claim 8, wherein, when the first and second jaws close, a non-linear longitudinal interface is defined along a length of the first and second jaws.

10. The end effector of claim 8, wherein, when the first and second jaws close, a non-linear lateral interface is defined along a width of the first and second jaws.

11. An end effector of a surgical tool, the end effector comprising:
a first jaw including first grips; and
a second jaw including second grips, the first and second jaws being transitionable between an open configuration and a closed configuration,
wherein, when the first and second jaws are in the closed configuration, the first and second grips mesh to define a first non-linear longitudinal interface along a first lateral side of the end effector.

12. The end effector of claim 11, wherein:
the first grips include first lateral grips longitudinally spaced apart to define first gaps therebetween; and
the second grips include first lateral grips longitudinally spaced apart to define second gaps therebetween,
wherein, when in the closed configuration, the first lateral grips of the first jaw are received in the second gaps, and the first lateral grips of the second jaw are received in the first gaps to define the first non-linear longitudinal interface.

13. The end effector of claim 12, wherein:
the first lateral grips of the first jaw each include a tissue contacting surface that vertically undulates toward and away from the second jaw; and
the first lateral grips of the second jaw each include a tissue contacting surface that vertically undulates toward and away from the first jaw.

14. The end effector of claim 12, wherein:
the first jaw includes a first lateral portion and a second lateral portion;
the first grips extend from the first lateral portion of the first jaw;
the second jaw includes a first lateral portion and a second lateral portion; and
the second grips extend from the first lateral portion of the second jaw.

15. The end effector of claim 14, wherein:
a first aperture is defined in the first jaw between the first and second lateral portions of the first jaw; and
a second aperture is defined in the second jaw between the first and second lateral portions of the second jaw.

16. The end effector of claim 14, wherein:
the first grips further include second lateral grips longitudinally spaced apart along the second lateral portion of the first jaw to define third gaps therebetween; and
the second grips further include second lateral grips longitudinally spaced apart along the second lateral portion of the second jaw to define fourth gaps therebetween,
wherein, when in the closed configuration, the second lateral grips of the first jaw are received in the fourth gaps and the second lateral grips of the second jaw are received in the third gaps to define a second non-linear longitudinal interface along a second lateral side of the end effector.

17. The end effector of claim 16, wherein:
the second lateral grips of the first jaw each include a tissue contacting surface that vertically undulates toward and away from the second jaw; and
the second lateral grips of the second jaw each include a tissue contacting surface that vertically undulates toward and away from the first jaw.

18. The end effector of claim 11, wherein:
the first grips include a first distal grip;
the second grips include a second distal grip; and
the first distal grip meshes with the second distal grip to define a first non-linear lateral interface along the width of the first and second jaws.

19. The end effector of claim 18, wherein:
the first grips further include a first proximal grip;
the second grips further include a second proximal grip; and
the first proximal grip meshes with the second proximal grip to define a second non-linear lateral interface along the width of the first and second jaws.
